(19) United States
(12) Patent Application Publication
(10) Pub. No.: US 2023/0255469 A1
(43) Pub. Date: Aug. 17, 2023

(54) AUXILIARY LIGHTING DEVICE FOR OPHTHALMIC SLIT LAMP

(71) Applicant: CHONGQING KANGHUA RUIMING SCIENCE TECHNOLOGY CO., LTD, Chongqing (CN)

(72) Inventor: Yi Wang, Chongqing (CN)

(21) Appl. No.: 18/004,053

(22) PCT Filed: Jan. 5, 2022
(86) PCT No.: PCT/CN2022/070185

§ 371 Date: Dec. 31, 2022

(30) Foreign Application Priority Data
Nov. 6, 2020 (CN) 202011227456.3

(51) Int. Cl. A61B 3/00; A61B 3/135

(52) U.S. Cl. CPC A61B 3/0008; A61B 3/135

(57) ABSTRACT

An auxiliary lighting device for ophthalmic slit lamp comprises a right lamp outer bracket, a right LED lamp, a right lamp inner bracket, a slit lamp large object lens tube, an objective lens base, a left lamp inner bracket, a left LED lamp, and a left lamp outer bracket; The large object lens tube of the slit lamp is installed on the objective lens base, the left end of the objective lens base is respectively equipped with the right lamp inner bracket and the left lamp inner bracket, and the large object lens tube of the slit lamp is located between the right lamp inner bracket and the left lamp inner bracket; The right LED lamp is installed on the top of the right lamp inner bracket, the right lamp outer bracket cover is located on the right lamp inner bracket, and the right LED lamp is located between the right lamp inner bracket and the right lamp outer bracket; The left LED lamp is installed on the top of the left lamp inner bracket, the left lamp outer bracket cover is located on the left lamp inner bracket, and the left LED lamp is located between the left lamp inner bracket and the left lamp outer bracket. The auxiliary lighting device is added to the new part to retain the auxiliary lighting function of the original part.

### TECHNICAL FIELD

The invention relates to the field of medical equipment, in particular to an auxiliary lighting device for ophthalmic slit lamp.

### BACKGROUND TECHNOLOGY

The eye is the most important organ in human senses, and about 80% of the knowledge in the brain is acquired through the eye. Reading, reading, painting, people and beautiful scenery all use eyes.

Slit lamp microscope is an indispensable important instrument for ophthalmic examination. It is also the first widely used basic instrument for routine ophthalmic examination. It is required by the state to configure slit lamp in ophthalmic diagnosis and treatment, ophthalmic health care, optometry and other industries

In the world, from the smallest ENT clinic to the largest ophthalmic hospital to the top ophthalmic disease research institution, the ophthalmic slit lamp microscope is the most professional equipment to examine the eyelid, conjunctiva, sclera, cornea, anterior chamber, iris, pupil, lens and the anterior 1/3 vitreous body, and determine the location, nature, size and depth of the lesions.

Ophthalmic slit lamp microscopes are widely used by medical institutions, ophthalmic health care institutions, and glasses chain stores. The expansion and improvement of the functions of the instruments play an important role in human eye health.

The structure of slit lamp is mainly composed of four parts: slit lighting mechanism, microscope optical body, mobile platform mechanism and cheek support mechanism.

At present, the slit lamp has an auxiliary lighting mechanism, which is installed on the following parts of the slit lamp:

1. The auxiliary lighting mechanism is installed around the reflector of the slit lighting mechanism, for example, on the upper part of the reflector of the slit lighting mechanism, and the light emitted shines on the eye surface through the emission mirror, or on the lower part of the reflector of the slit lighting mechanism, and the light emitted directly slants to the eye surface;

2. The auxiliary lighting mechanism is installed in the left and right rotating shaft holes of the crack lighting mechanism, and the light emitted directly slants onto the eye surface.

These auxiliary lighting devices have the following disadvantages:

1. It is installed on the crack lighting mechanism. When using, the crack lighting mechanism should be adjusted to the left and right so that the auxiliary lighting light can reach the required position. It is inconvenient to use. For example, if the optical body is moved to the left and right, the lighting mechanism should be moved to the left and right so that the light can reach the inspection position evenly.

2. Whether the light is reflected by the reflector or directly illuminated, it is illuminated from one direction (equivalent to a point light source). The light is easily blocked by the objects in front, causing uneven illumination, such as eyelashes.

3. The slit lamp also needs more auxiliary light of different wavelengths for illumination, and needs to increase the position.

### SUMMARY OF THE INVENTION

In view of the shortcomings of the prior art, the invention adds an auxiliary lighting device to the slit lamp, which can solve the problem of multi angle illumination of auxiliary lighting as the slit lamp moves. Aiming at the above problems, an ophthalmic slit lamp auxiliary lighting device is proposed. The specific technical scheme is as follows:

An auxiliary lighting device for ophthalmic slit lamp includes an objective lens base, which is characterized in that it also includes a bracket and a lighting lamp, wherein a bracket is arranged on the objective lens base, and a lighting lamp is arranged on the bracket.

Furthermore, the two brackets are respectively arranged on both sides of the end face of the objective lens base, the large object lens barrel of the slit lamp is located between the two brackets, and each bracket is provided with a lighting lam.

Furthermore, the lighting lamp is an LED lamp.

Furthermore, the wiring of the lighting lamp is hidden inside the objective lens base.

The beneficial effects of the invention are:

First, the structure is simple and easy to realize, and the light line is also easy to hide in the body;

Second, the auxiliary lighting device is set on both sides of the optical volume microscope for observation, with more irradiation angles and wider positions, and can also follow the optical volume when it rotates left and right.

Third, the auxiliary lighting device is added to the new part to retain the auxiliary lighting function of the original part.

### DESCRIPTION OF ATTACHED DRAWINGS

FIG. 1 is the structure diagram of the patent;

FIG. 2 is the sectional view of the patent structure;

FIG. 3 is an exploded view of the patent structure;

The attached figure in the figure shows that the right lamp outer bracket 1, the right LED lamp 2, the right lamp inner bracket 3, the slit lamp large object lens tube 4, the objective lens base 5, the left lamp inner bracket 6, the left LED lamp 7, and the left lamp outer bracket 8.

### SPECIFIC EMBODIMENTS

The preferred embodiments of the invention are described in detail below in combination with the accompanying drawings, so that the advantages and features of the invention can be more easily understood by those skilled in the art, and the scope of protection of the invention can be more clearly defined.

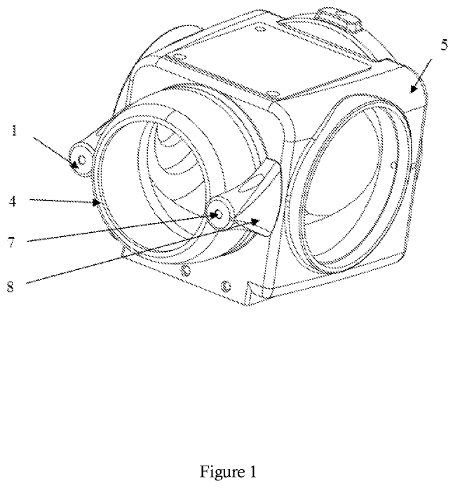

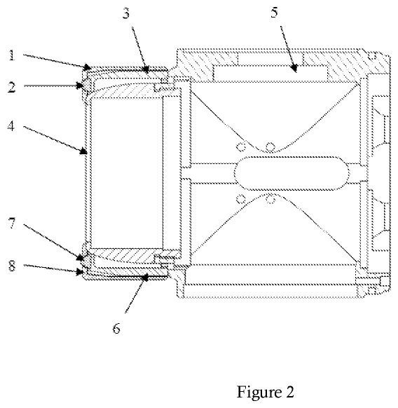

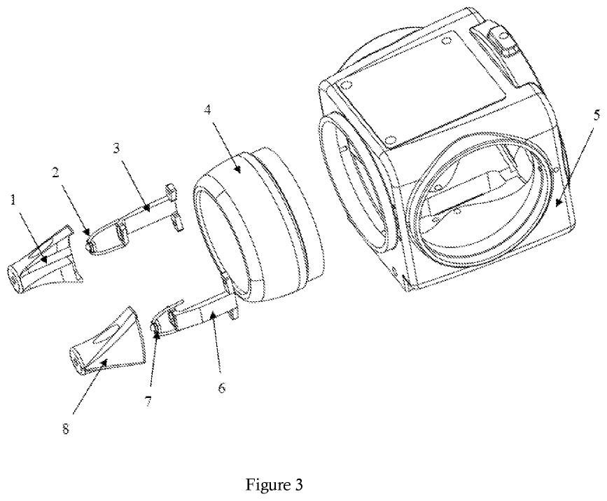

The implementation of the invention, for example, is shown in FIGS. 1 to 3: an ophthalmic slit lamp auxiliary lighting device, including a right lamp outer bracket 1, a right LED lamp 2, a right lamp inner bracket 3, a slit lamp large object lens tube 4, an objective lens base 5, a left lamp inner bracket 6, a left LED lamp 7, and a left lamp outer bracket 8;

The large object lens tube 4 of the slit lamp is installed on the objective lens base 5. On the left end surface of the objective lens base 5, the right lamp inner bracket 3 and the left lamp inner bracket 6 are respectively installed. The large object lens tube 4 of the slit lamp is located between the right lamp inner bracket 3 and the left lamp inner bracket 6;

The right LED lamp 2 is installed on the top of the right lamp inner bracket 3, the cover of the right lamp outer bracket 1 is located on the right lamp inner bracket 3, and the right LED lamp 2 is located between the right lamp inner bracket 3 and the right lamp outer bracket 1;

The left LED lamp 7 is installed on the top of the left lamp inner bracket 6, the cover of the left lamp outer bracket 8 is located on the left lamp inner bracket 6, and the left LED lamp 7 is located between the left lamp inner bracket 6 and the left lamp outer bracket 8.

For those skilled in the art, it is obvious that the invention is not limited to the details of the above exemplary embodiments, and can be realized in other specific forms without departing from the spirit or basic features of the invention. Therefore, from any point of view, the embodiments should be regarded as exemplary and non restrictive. The scope of the invention is defined by the appended claims rather than the above description. Therefore, it is intended to include all changes within the meaning and scope of the equivalent elements of the claims in the invention. Any reference mark in the claims shall not be regarded as limiting the claims involved.

In addition, it should be understood that although the Description is described according to the embodiments, not every embodiment only contains an independent technical solution. This description of the Description is only for the sake of clarity. Those skilled in the art should take the Description as a whole. The technical solutions in each embodiment can also be properly combined to form other embodiments that can be understood by those skilled in the art.

1. An auxiliary lighting device for ophthalmic slit lamp includes an objective lens base, which is characterized in that it also includes a bracket and a lighting lamp, wherein a bracket is arranged on the objective lens base, and a lighting lamp is arranged on the bracke.

2. The auxiliary lighting device for ophthalmic slit lamp according to claim 1, which is characterized in that: two said brackets are respectively arranged on both sides of the end face of the objective lens base, the large object lens barrel of the slit lamp is located between the two said brackets, and each said bracket is provided with a lighting lamp.

3. The auxiliary lighting device for ophthalmic slit lamp according to claim 1, which is characterized in that the lighting lamp is an LED lamp.

4. The auxiliary lighting device for ophthalmic slit lamp according to claim 1, which is characterized in that the wiring of the lighting lamp is hidden inside the objective lens base.
